(19) United States
(12) Patent Application Publication
(10) Pub. No.: US 2022/0367013 A1
(43) Pub. Date: Nov. 17, 2022

(54) SYSTEM AND METHOD FOR DIGITALIZATION, ANALYSIS AND STORAGE OF BIOLOGICAL SAMPLES

(71) Applicant: MANTISCOPE TIBBI CIHAZLAR ARASTIRMA VE GELISTIRME LTD. STI., Ankara (TR)

(72) Inventors: Cem COTELI, Ankara (TR); Goksu GURER, Ankara (TR)

(73) Assignee: MANTISCOPE TIBBI CIHAZLAR ARASTIRMA VE GELISTIRME LTD. STI., Ankara (TR)

(21) Appl. No.: 17/623,276

(22) PCT Filed: Sep. 2, 2019
(86) PCT No.: PCT/TR2019/050718

§ 371 Date: Dec. 28, 2021

(30) Foreign Application Priority Data
Jul. 1, 2019 (TR) 2019/09740

(51) Int. Cl. G16H 10/40; G01N 35/00; G02B 7/36

(52) U.S. Cl. CPC G16H 10/40; G01N 35/00029; G01N 35/00732; G01N 35/00871; G02B 7/36

(57) ABSTRACT

Biological samples are prepared on a slide for physician or veterinarian interpretation in a case of a diagnosis for human or animal diseases. The biological samples are specimens taken from certain areas or body fluids of human or animal. The biological samples are placed on the slide and are made ready for an examination without any process or after physical processes. Physicians or veterinarians diagnose by interpreting the biological samples over a microscope. Digitizing data, usage of data processing techniques and automatic reporting are activities reducing a workforce of expert physicians or veterinarians with a developing technology by abandoning manual methods. As digital pathology and hematology are main technical fields, a system and integrated methods about digitizing, analyzing and storing biological samples are given.

### TECHNICAL FIELD

Biological samples are prepared on the slide for physician or veterinarian interpretation in the case of the diagnosis for human or animal diseases. These samples may be specimens taken from certain areas or body fluids of human or animal. The samples can be placed on the slide and are made ready for the examination without any process or after physical processes (washing, dyeing, etc.). Physicians or veterinarians diagnose by interpreting these samples over a microscope. Digitizing the data, usage of data processing techniques and automatic reporting are activities that reduce the workforce of expert physicians or veterinarians with the developing technology by abandoning manual methods. As digital pathology and hematology are the main technical fields of the presented invention; system and integrated methods about digitizing, analyzing and storing biological samples are given within the scope of the invention.

### STATE OF THE ART

Digital pathology and hematology are studies that digitize the examination activities of a physician or veterinarian with a manual microscope.

The methods of collection, analysis and storage of biological data are evaluated within the scope of these studies; There is a need for a compact and cost-effective solution that can handle to solve these problems.

Cost increasing factors can be listed as follows.
The difficulty of collecting data and the dependence over the individuals, costs of microscope and camera.
Data can not be stored in a sequential structure (sample images, patient and doctor relationship, diagnosis) and it is not easily accessible.
High cost for data processing hardware.

As there are microscopes which are capable of only automatic scanning and data collection, there are information systems which are only used to store data in a hierarchical structure and there are methods which are only used for data processing; these cost effective solutions are required to be placed on the market.

As a result, introducing cost-effective solutions in the market will enable the systems to be extended to the smallest healthcare institutions and provide a compact environment where physicians, veterinarians and data collection equipment are combined for the big data.

The patent application US20070014460A1 in the state of the art mentions a method after the acquisition of peripheral blood smear images as the separation of the parts and the categorization of each seperated part by an analysis method. In this way, it mentions that the method is able to separate blood cells in peripheral blood smear images and then relate them to specific types.

U.S. Pat. No. 4,362,386A discloses a method with a hardware including the process of counting red and white blood cells at a desired level via the peripheral blood smear. Counting results are displayed on a monitor.

U.S. Pat. No. 4,741,043A proposes a method about how to automate a manual microscope. This publication which is published in a general level, is presented with the flow which is necessary to collect a certain number of images on the slide.

U.S. Pat. No. 5,812,419A specifies a mechanical model describing the preparation of the biological slide using one drop of blood and subsequent analysis after the corresponding preparations.

U.S. Ser. No. 10/223,502B2 mentions an established cloud system design for the examination of biological samples. It mentions that the data will be examined in this cloud system and made available to the user.

US20180211380A1 proposes a method for examining cell samples using machine learning. The images taken by means of a hardware are processed with the help of machine learning. Segmentation and identification methods are described in this publication.

US20180060993A1 includes a method for collecting images using motorized structures attached to a microscope and subsequent analysis using machine learning methods.

EP0628822A2 describes a fully automated preparation test containing biological samples from one drop of blood.

EP2083268A1 proposes a method for counting 5 different types of blood cells from a blood sample.

JPH0720124A also proposes an automated microscope system for the examination of blood samples.

WO2018211418A1 proposes a method for the selection of the region in the peripheral blood smear sample to be screened. It is important for the analysis to select a specific region instead of looking at the entire slide surface.

WO2019102277A1 also proposes a method for the counting parameters required in hematology samples. This method can be used in the evaluation of hematological diseases.

JP2016520806A proposes a method for counting blood cells by optical methods using the fluidity of blood.

### Brief Description of the Invention

Biological samples are examined by the physicians or veterinarians and then these samples are disposed or physically stored.

The test results are expressed either in verbal or written form to the patient or the person who is associated with the patient and the results are kept in an information system.

This condition prevents objective evaluation, physician-related errors would occur, and delays the relevant diagnosis due to non-digitized data.

For the evaluation by a new physician, it is necessary to re-evaluate the relevant sample manually and to re-sample the patient or animal if there is no sample.

The presented invention proposes system and methods for digitizing biological samples, analyzing them with an auxiliary data processing method, storing, and allowing the physician to achieve results easily.

The invention, is a combined system which contains cloud-based artificial intelligence and cloud-connected slide scanners that are used to scan microscope ready biological samples and analyse with the help of data processing.

By means of the invention, a new generation digitalization method has been proposed, and an example method is given for the examination and digitization of peripheral blood smears which are used by hematologists and pathologists frequently.

In the detailed description of the invention, peripheral blood smear is given as an artificial intelligence algorithm, which is an example of a data processing algorithm. The same flow can be used by adding the relevant algorithm to the examination of other biological samples.

### FIGURES THAT ARE HELPFUL TO UNDERSTAND THE INVENTION

FIG. 1: Structure of the centralized cloud system with multiple hardware

FIG. 2: Hardware used for digitizing the biological sample images

FIG. 3: Digitizing using the slide scanner hardware

FIG. 4: Cloud flow diagram

FIG. 5: Hardware's technical drawings from the front view

FIG. 6: Hardware's technical drawings from the right view

FIG. 7: Hardware's technical drawings from the back view

FIG. 8: Hardware's technical drawings from the isometric view

FIG. 9: Focusing slices—Dividing large areas into smaller segments, noise and active image ranges

FIG. 10: Separation of noise and active image segments after convergence

FIG. 11: Peripheral blood smear analysis method blocks

FIG. 12: Parsing block subcomponents

FIG. 13: Artificial Neural Network with its defined inputs and outputs

FIG. 14: Testing the results coming from the Artificial Neural Networks

FIG. 15: Testing the results with the Jaccard Index

FIG. 16: Data analysis and subsequently storage

### DETAILED DESCRIPTION OF THE INVENTION

The system consists of hardware and software components.

The hardware component includes XYZ axis motors, motor controller, microscope light, zeroing switches, CCD camera, ocular, immersion oil dripping system, automatic lens changer, and Internet of Things (IoT) microprocessor (C1.13).

The hardware is connected to the cloud software with the IoT password provided from the user accounts registered in the cloud. The method to obtain this password is described in the Cloud-C2 section.

The software component consists of 3 different software elements that communicate with each other.

The first software element is an embedded software built on the Internet of Things (IoT) microprocessor (C1.13), which is used to automate the data processing process.

The second software element is an item to enable the user to review the images acquired by the hardware and it is installed to provide an interface between the hardware and the user over the cloud.

At the same time, this software is a web page that allows users to register and opens test reports coming from the hardware devices which are associated within the same institution to the registered users. This interface is described in detail in the Cloud-C2.

The third software element is an algorithm service that includes an artificial intelligence algorithm which takes an image as input and gives the results of the artificial intelligence analysis. This interface is described in detail in the Cloud-C2.

The system is designed to be cost-effective to reduce costs for the examination of patient or animal samples in small budget health care facilities and accelerate the process. The designs of the present invention are considered according to the cost-effective system and should be considered in this context.

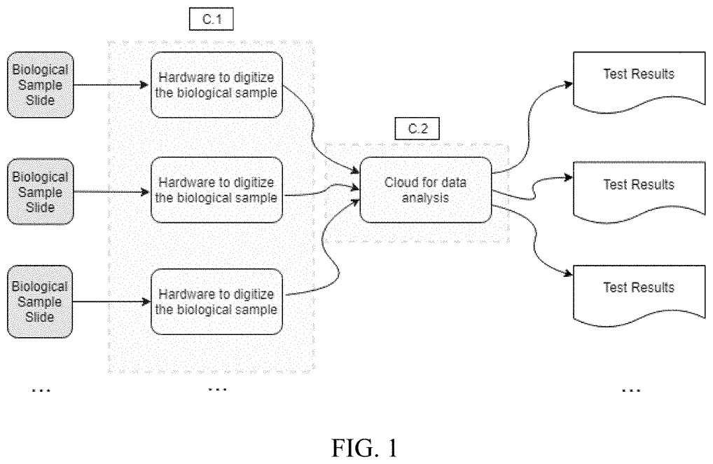

The system can be divided into two components, C1 (hardware for digitizing the biological samples) and C2 (cloud for data analysis). The general system level block diagram is given in FIG. 1.

4.1 Hardware—C1

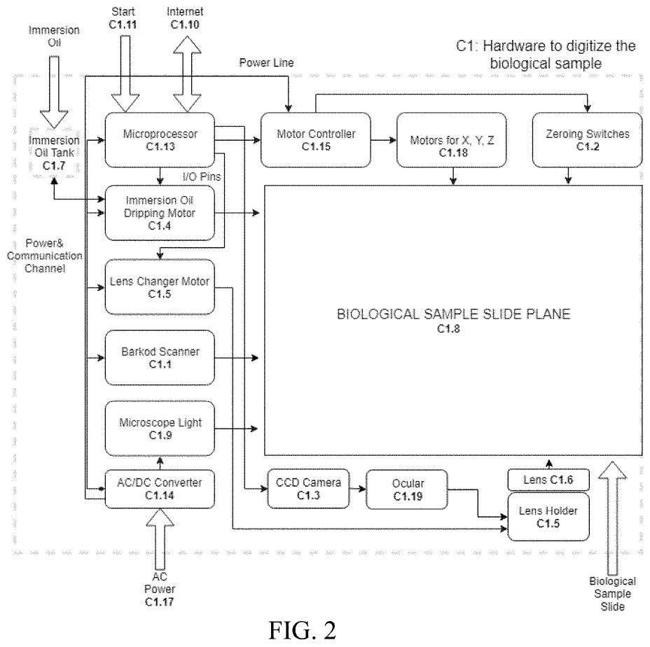

The diagram which shows the hardware components inside C1 can be seen in FIG. 2.

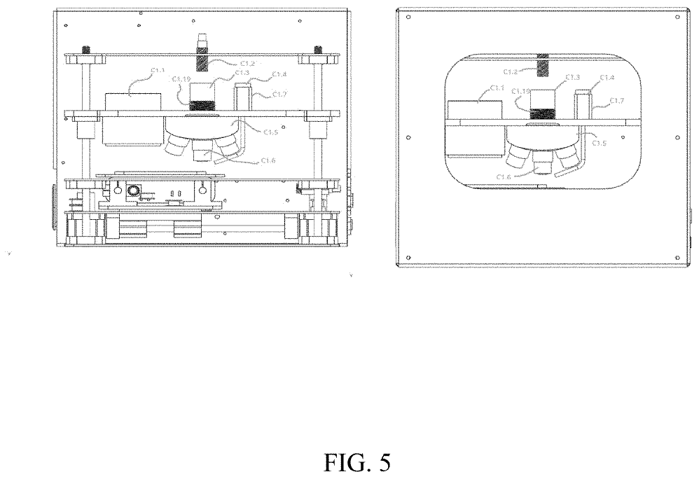

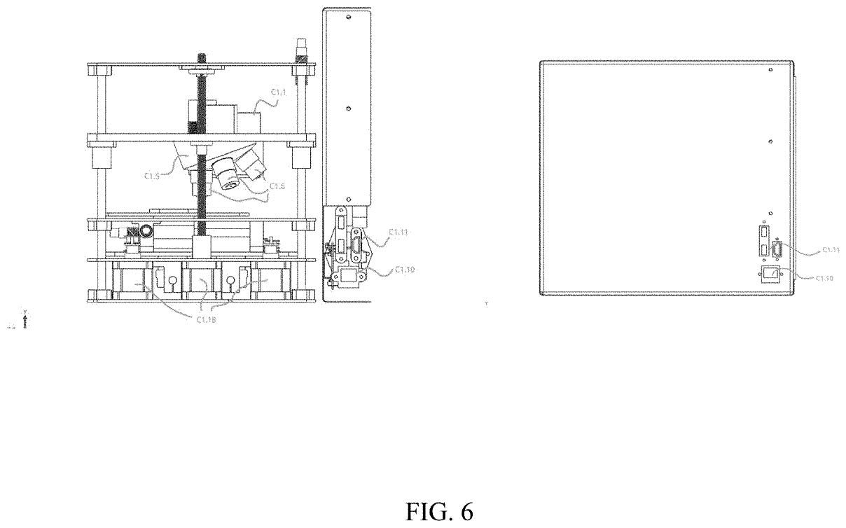

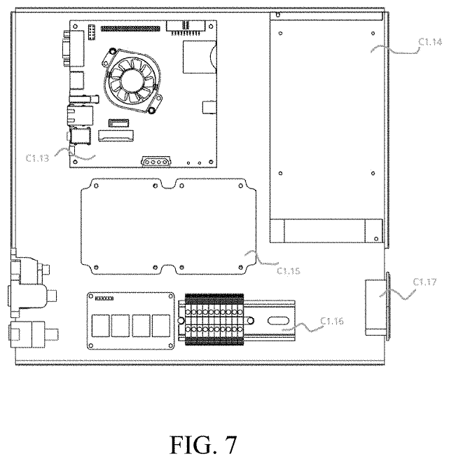

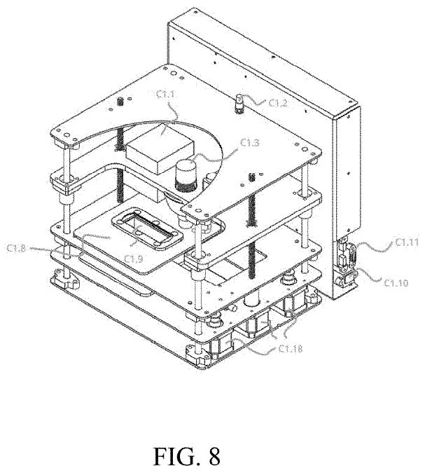

Detailed technical drawings (C1) of the equipment are shown in FIGS. 5, 6, 7, 8 as in closed and open forms.

C1.13 is the main control unit for all hardware components in the hierarchy to digitize the biological sample.

The inputs of the hardware system are the biological sample, AC power, immersion oil tank and starting switch.

The data interface between the system and the cloud is wired or wireless Ethernet.

A barcode can be generated for the relevant human or animal from the interface on the cloud with the patients recorded over the cloud. This barcode allows the hardware to digitize and send biological samples to the cloud without knowing any specific information about humans or animals.

After inserting the biological sample with the printed barcode affixed on it to the device or manually scanning the barcode under the C1.1 Barcode scanner and placing the biological sample, the data collection process starts by pressing the ‘Start’ button.

The biological sample inspection procedure depends on the type of sample.

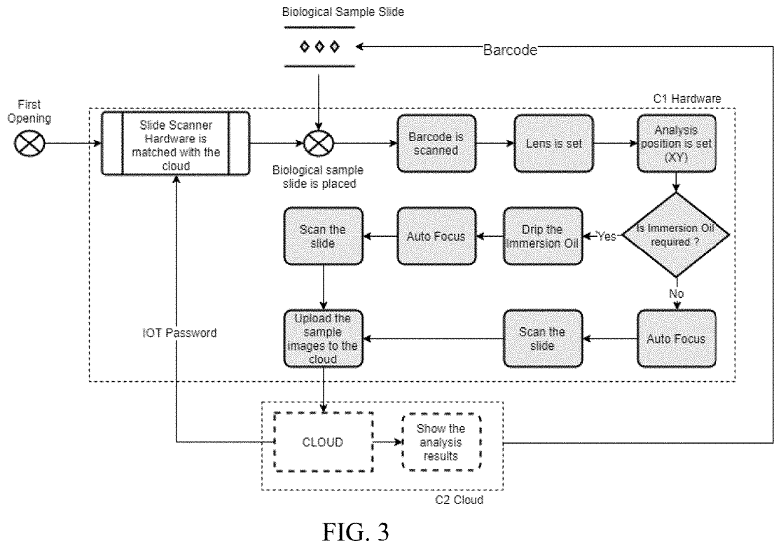

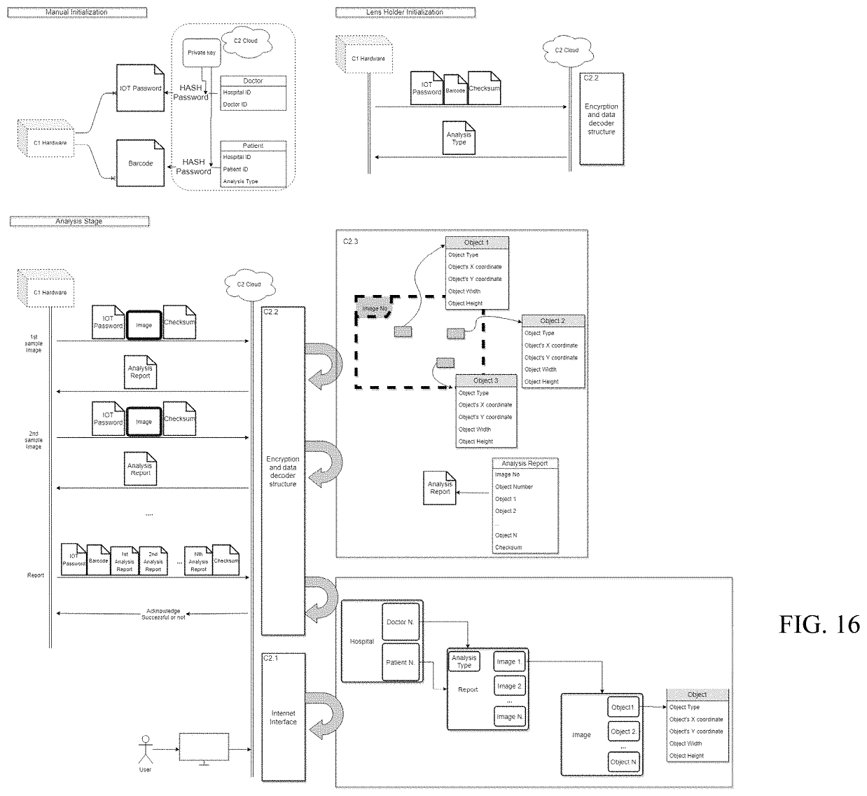

The type of sample is used to decide on the magnification levels. The system has different magnification levels such as 10×, 40× and 100×. For example; a minimum of 10× magnification and a single image is sufficient for the analysis of Thoma slides. However, peripheral blood smear (PBS) slides require a magnification level of 100× with immersion oils to analyze blood cell disorders and multiple images. The first software element is involved at this stage. The first software item that reads the barcode sends the barcode information to the Cloud as defined in FIG. 16. After the biological sample type and the number of samples that need to be collected have arrived via the cloud, the data collection function from the biological sample starts. The analysis type can also be changed by the user in manual operation (usage) mode. The flow block diagram can be followed from FIG. 3.
1. The slide scanner hardware is associated with the cloud using the IoT password during startup/initiation.
2. A barcode can be generated for the relevant human or animal from the interface on the cloud with the patient records available on the cloud. This barcode is printed out from the cloud and pasted onto the sample.
3. The user presses the Start button.
4. C1.13 moves the sample slide plate in the direction of the barcode scanner using the XY movement and the barcode is scanned using the barcode scanner. If the barcode information is not pasted on the sample, it can also be read by hand (manually) under the barcode scanner. The information is sent to C1.13. FIG. 16 gives details about how the analysis type and sampling the number of images information is received over the barcode.
5. C1.13 decides whether 100× magnification is required or not depending on the biological sample type. Multiple or single image acquisition requirements are also updated using the barcode information.
6. C1.13 sends a request to the C1.5 Lens holder to adjust the lens to the specified magnification level using motorized operations.
7. C1.13 moves the slide plate to a new XY position for data collection.
8. C1.13 sets the Z axes to the auto focus starting position.
9. Case 1: Single Image mode, no 100× magnification.
   Auto Focus: C1.13 captures images from the C1.3 camera to calculate sharpness and changes the Z position.
   C1.13 captures the image after the auto focus is completed.
10. Case 2: Single Image mode, with 100× magnification.
   C1.13 sends the request to the C1.4 immersion oil dripper. Immersion oil is dripped over the sample.
   Auto Focus: C1.13 captures images from the C1.3 camera to calculate sharpness and changes the Z position.
   C1.13 captures the image after the auto focus is completed.
11. Case 3: Multiple image mode, no 100× magnification.
   Auto Focus: C1.13 captures images from the C1.3 camera to calculate sharpness and changes the Z position.
   C1.13 captures the image after the auto focus is completed.
   C1.13 moves the sample slide plate to different XY positions for data processing, depending on the desired number of image samples. Images are taken for each position.
12. Case 4: Multiple image mode, with 100× magnification.
   C1.13 sends the request to the C1.4 immersion oil dripper. Immersion oil is dripped over the sample.
   Auto Focus: C1.13 captures images from the C1.3 camera to calculate sharpness and changes the Z position.
   C1.13 captures the image after the auto focus is completed.
   C1.13 moves the sample slide plate to different XY positions for data processing, depending on the desired number of image samples. Images are taken for each position.
13. C1.13 sends images obtained for data processing to the cloud by inserting the barcode information.
   The Y-axis in the flow is given as the Z-axis in FIGS. 5, 6, 7 and 8; The Z axis is also referred to as the Y axis. The next steps are described in Cloud-C2.

4.1.1 Auto Focus Method

The automatic focus of biological samples in a cost-effective system without position feedback for the Z axis on the magnification levels of 10×, 40× and 100× is a problem that is studied by engineers. The system, which is designed for laboratories and small health institutions, has to have the ability to perform autofocus without user interaction at all magnification levels, since it is a necessary feature for the entire steps.

The invention proposes a method by changing the lens position on the Z axis and performing auto focus, by controlling the camera outputs C1.4. This method is contained in the software block installed in the biological sample digitization hardware (C1). The system does not have a position feedback which shows whether the motor controller applies movement order given by C1.13 or not in 5 μm precision for the Z axes by considering the cost-effective system has a 5 μm precision.

In case of small position changes, system control cannot be achieved in the specified direction as desired. However, the system can control the position on the Z-axis at a position movement greater than 200±5 μm. The errors in this stage are not regarded.

The procedure, scans the aforementioned region between Zₗ and Zₕ in 5 μm slices twice times, the reality accepted at this stage is defined with the expression Zₕ≥Zₗ+200 μm. The first scan is used to extract noise and active zone Gaussian distributions. The second scan is used to focus the sample using the gauss distributions calculated in the first scan.

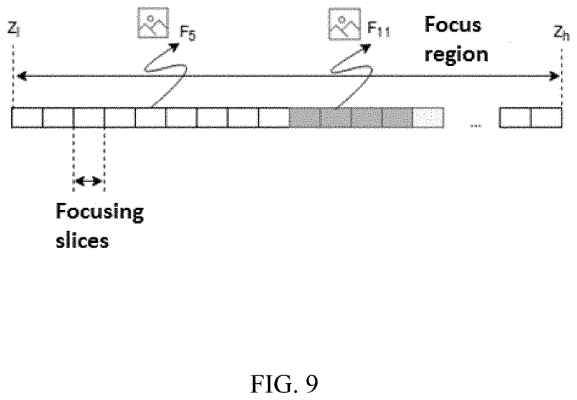

The procedure, defines the first scan to find K=2 number of Gaussians, divides the region into 5 μm slices with the number of N>40 samples. The scanning iterations can be seen from FIG. 9.
The system assumes that the position to be focused on distance ranges are in the range between Zₗ and Zₕ.
The sharpness of the image taken from the camera is the focus parameter of system. W is used for the width of the image and also H is used for the height of the image. The sharpness is calculated using the following formulas.

$$S ( x , y ) = \frac{{\partial }^{2} I ( x , y )}{\partial {x}^{2}} + \frac{{\partial }^{2} I ( x , y )}{\partial {y}^{2}} \qquad ( 1 )$$

$$μ = {\sum }_{x = 1}^{x = W} {\sum }_{y = 1}^{y = L} \frac{1}{W * L} S ( x , y ) \qquad ( 2 )$$

$$σ = \sqrt{\frac{1}{W * L} {\sum }_{x = 1}^{x = W} {\sum }_{y = 1}^{y = L} {[ S ( x , y ) - μ ]}^{2}} \qquad ( 3 )$$

The Fₗ focus parameter is calculated as σ². This parameter defines how the edge-based components are dominant on the image.
The aim is to find the Gaussian model of the focal parameter calculated from the sequence of images in which biological objects are located when the position changes.
The focus parameters between the region Zₗ and Zₕ will be used in Gaussian modelling for the sharpness function of the image that is meaningless and contains parasitic with lacking a biological object in some regions and the active region containing the view. During the first scan, the focus parameters will be collected and the data set required for training will be created.

To find the centroids of Gaussian clusters, the data is divided into K=2 for noise and active zone. The procedure is:
(a) K points are randomly selected from the data set. They are defined as cluster centers.
(b) The distance between each element of the data and the centers of the previously defined clusters is found. For example, let's take two sampling points F₁ and F₂, the distance between these two points is calculated as follows:

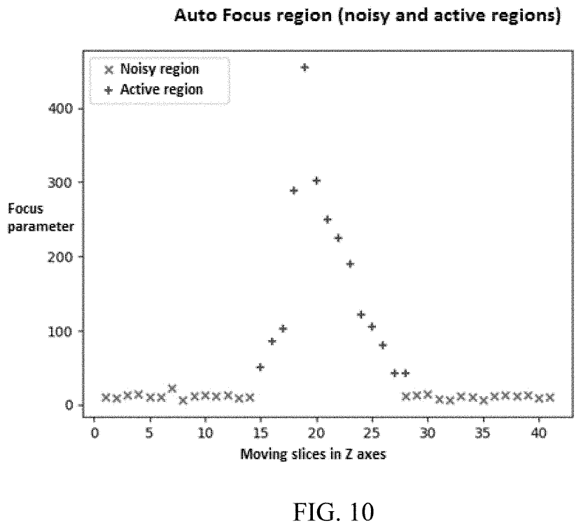

D=\sqrt{F₁²−F₂²} (4)
(c) Sampling points are assigned to the nearest centroid according to the distance formula.
(d) The average value of each cluster is calculated by averaging the samples associated with each cluster.
(e) Items between b-d are repeated until the convergence point.
The clustered regions contain a data set that is distinguished from each other as in FIG. 10.
In the first scan, the automated microscope understands the active and noise zones. Each set was then modeled as Gaussian distributions.

$${μ}_{n} = \sum \frac{1}{{K}_{n}} {F}_{n} , {μ}_{a} = \sum \frac{1}{{K}_{a}} {F}_{a} \qquad ( 5 )$$

Fₙ is the cluster containing noise zone elements, Fₐ is the cluster containing active zone elements, Kₙ is the number of elements in the noise zone, and Kₐ is the number of elements in the active zone.

$${σ}_{n} = \sqrt{\frac{1}{{K}_{n}} \sum {[ {F}_{n} - {μ}_{n} ]}^{2}} , {σ}_{a} = \sqrt{\frac{1}{{K}_{a}} \sum {[ {F}_{a} - {μ}_{a} ]}^{2}} \qquad ( 6 )$$

The following procedure describes the conditions required to find the focusing position during the second scan using a trained structure by the data set obtained in the first scan.
Focusing procedure starts from Zₗ to repeat in 5 μm slices.
The focus parameter (Fᵢ) is associated with noise (Gₙ), active (Gₐ), and peak regions (P) according to the following conditions. When condition P is fulfilled, the system is in the autofocus position condition.

Fᵢ≤μₙ+3*σₙ,Gₙ (7)

μₙ+3*σn≤Fᵢ≤μₐ+3*σₐ,Gₐ (8)

μₐ+3*σₐ≤Fᵢ,P (9)
The system stops at that position and C1.3 CCD camera is used to capture image.
The procedures after this step are the operations for the acquisition of the desired number of images in the horizontal plane and uploading the images to the cloud.

4.2 Cloud—C2

Cloud computing is a powerful tool of choice with the advantages of rapid adaptation for the algorithms and the ability to connect the hardware via the Internet over the cloud that have a physical IP and address.

Installing data processing methods on each hardware causes cost-increasing results and makes software updates difficult. Therefore, this invention has taken the burden of data processing methods from the hardware and draws it to a central area that all connected hardware can use. In this way, hardware can be produced in a cost-effective manner and also software updates can be made fast and a safe working environment can be provided.

The cloud system in the mentioned invention, connects the slide scanner hardwares that are used to scan the biological samples to the central system. In this way, data coming from hardware installed in different environments can be synthesized and archived.

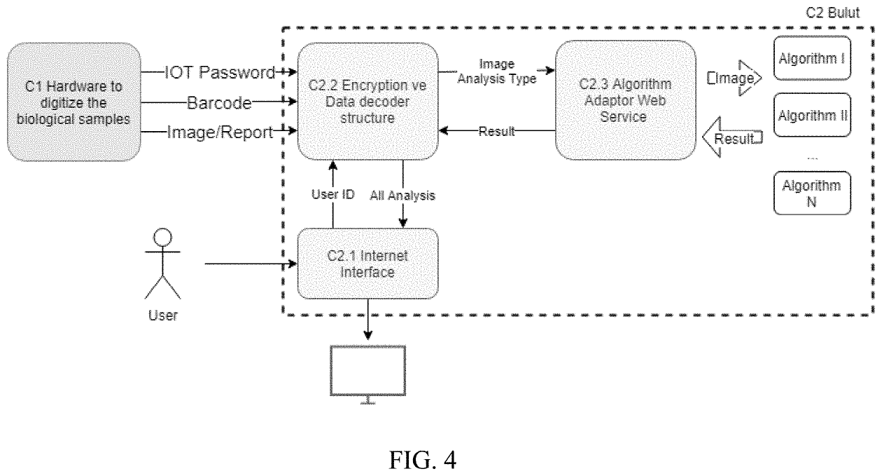

There are two software elements in the cloud system. The first one includes the C2.1 Web interface and the C2.2 encryption and data decoder structure, and the other one is the software component called as C2.3 algorithm adapter web service, which plays a role in system administration. The processes in the cloud (C2) object are examined in FIG. 4.

Hardware-Acquired Images:
1) The biological sample image and data processing type are sent to the cloud system “Encryption and data decoder structure” with the hardware C1.
2) C2.2 “Encryption and data decoder structure” directs the biological sample image to C2.3 “Algorithm Adapter Web Service” according to the data processing type.
3) C2.3 “Algorithm Adapter Web Service” runs the data processing method for the relevant biological sample image and the results are taken directly from the algorithm block.
4) C2.2 “Encryption and data decoder structure” sends the results to the hardware for the corresponding biological sample image.
5) Hardware (C1) repeats steps 1-4 for each biological sample image. When data processing of all images is finished, the hardware (C1) sends the batch results with the barcode data together to C2.2 “Encryption and data decoder structure”. The data flow can be followed in FIG. 16.
6) C2.2 “Encryption and data decoder structure” sends all collected results to user approval in C2.1 “Web Interface”.
7) After user approval, the data processing results of the batch images are made available to the user on the cloud (C2).

Images Taken Manually by the User:
1) The user enters the Cloud (C2) with a password and a user name.
2) The user selects the data processing type and the patient to associate.
3) The user uploads the biological sample images to the system via a web page.
4) The biological sample image and the data processing type are sent to the cloud system C2.2 “Encryption and data decoder structure” via C1.2 the Web Interface.
5) C2.2 “Encryption and data decoder structure” directs the biological sample image to C2.3 “Algorithm Adapter Web Interface” according to the data processing type.
6) C2.3 “Algorithm Adapter Web Service” runs the data processing method for the relevant biological sample image and the results are taken directly from the algorithm block.
7) C2.2 “Encryption and data decoder structure” sends the results to the C2.1 “Web Interface” for the corresponding biological sample image.
8) C2.1 “Web Interface” repeats steps 4-7 for each biological sample image. When all images are finished, it sends the batch results to the C2.2 “Encryption and data decoder structure” with the associated patient information.
9) C2.2 “Encryption and data decoder structure” sends all collected results to user approval in C2.1 “Web Interface”.
10)After user approval, the data processing results of the batch images are made available to the user on the cloud (C2).

As these steps are applied for digitizing and data processing of biological samples; different data processing methods can be used for different types of biological samples. As an example; A data processing method installed on the system for peripheral blood smear samples is described below.

4.2.1 Example of Biological Sample Data Processing Algorithm

Peripheral Blood Smear is frequently used test by hematology and pathology departments for the diagnosis leukemia, anemia and thalassemia with the help of expert physician. This test is used in the form of one drop of blood taken from the person on the slide, after smearing, staining and washing, and the diagnosis is made by the expert physicians after the examination under the microscope.

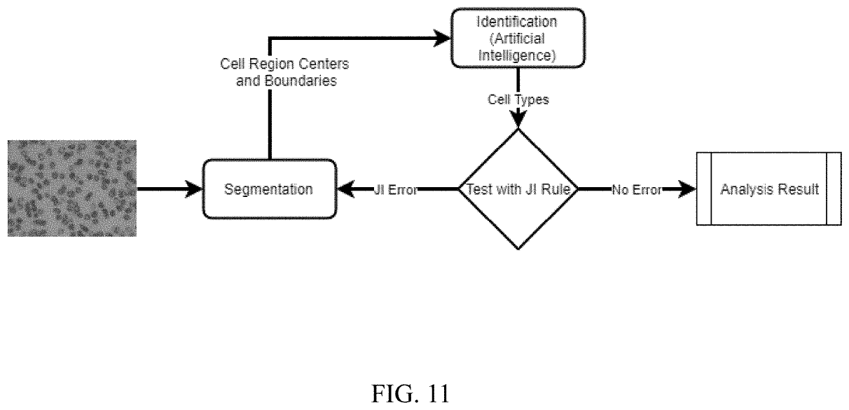

The invention proposes a method for digitizing biological samples, which provides a peripheral blood smear data processing method as a sample algorithm data processing method. This method uses 3 different blocks for data processing of the acquired biological sample images. These blocks are segmentation, identification and correcting blocks. These blocks are shown in FIG. 11.

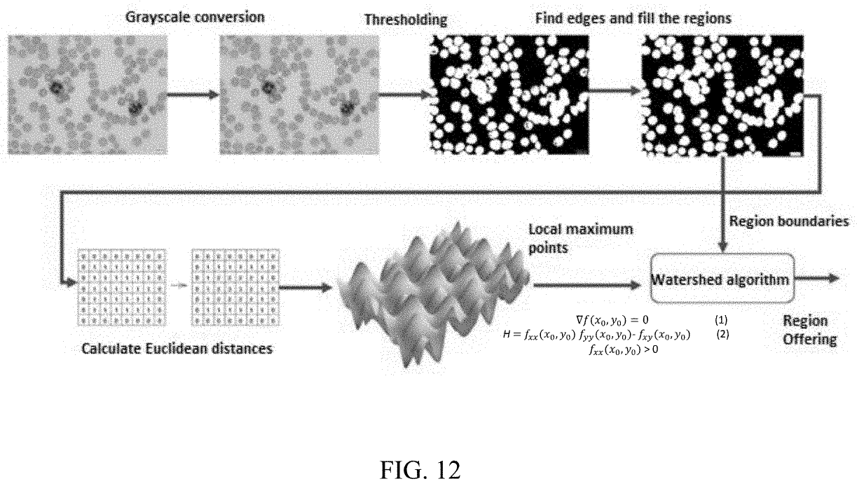

Separation block can be examined with 6 different sub-components. These sub-components work for a 2-dimensional 3-color image; gray scale conversion, thresholding, edge detection, euclidean distance calculation, local maximum points and watershed algorithms. These sub-components are shown in FIG. 12; and their mathematical models are also described below.

$$Gray scale conversion I ( x , y ) = \frac{1}{3} * [ I ( x , y , R ) + I ( x , y , G ) + I ( x , y , B ) ] \qquad ( 10 )$$

$$Thresholding ; v is threshold value . If : I ( x , y ) > v , I ( x , y ) = 1 ; \\ Else : , I ( x , y ) = 0 \qquad ( 11 )$$

$$Edge detection I ( x , y ) = \frac{{\partial }^{2} I ( x , y )}{\partial {x}^{2}} + \frac{{\partial }^{2} I ( x , y )}{\partial {y}^{2}} \qquad ( 12 )$$

$$Euclidean Distance Transform If : I ( {x}_{k} , {y}_{k} ) = 1 ; {\to }^{minimum [ D ]} I ( {x}_{l} , {y}_{l} ) = 0 ; \qquad ( 13 )$$

$$D = \sqrt{{( {x}_{k} - {x}_{l} )}^{2} + {( {y}_{k} - {y}_{l} )}^{2}} \qquad ( 14 )$$

$$Detection of Local Maximum Points ∇ I ( x , y ) = [ \frac{{\partial }^{2} I ( x , y )}{\partial {x}^{2}} , \frac{{\partial }^{2} I ( x , y )}{\partial {y}^{2}} , \frac{{\partial }^{2} I ( x , y )}{\partial x * \partial y} ] = [ {f}_{xx} , {f}_{yy} , {f}_{x y} ] \qquad ( 15 )$$

$$H = {f}_{x x} * {f}_{y y} - f x y \qquad ( 16 )$$

$$If : H > 0 , {f}_{x x} > 0 ; Local Maximum Watershed Algorithm \qquad ( 17 )$$

(a) The pixels to which the local maximum points belong represent the center for a cell. Starting from these pixels, the places where the pixel values are 1 are marked.
(b) Object segmentation is ended where the pixel value is 0, the region where the cell is located is detected.

The output of the segmentation block results the regions defined for each cell. These regions refer to an object but there is no information about the type of the cell.

The identification block contains a trained artificial intelligence. This artificial intelligence has extracted the features of the images for each block for 20000 different cell samples, by using convolutional neural network (CNN) blocks. The last value indicates the cell type. These steps were introduced to the system by expert physicians.

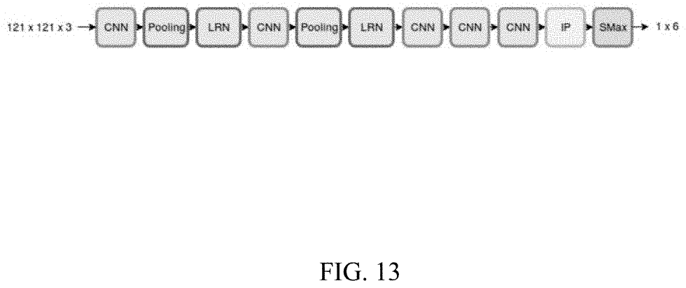

For the object identification in the defined region, the result of the artificial intelligence is obtained. The block of artificial neural networks used for this step is described in FIG. 13.

The correction block is a result correction process for the output of segmentation and identification blocks. For each cell region, Jaccard index (JI) is used to test its relationship with other regions. JI can be expressed as follows. For example, the JI value between zone A and zone B is calculated as follows.

$$J ( A , B ) = \frac{A ⋂ B}{A ⋃ B} \qquad ( 18 )$$

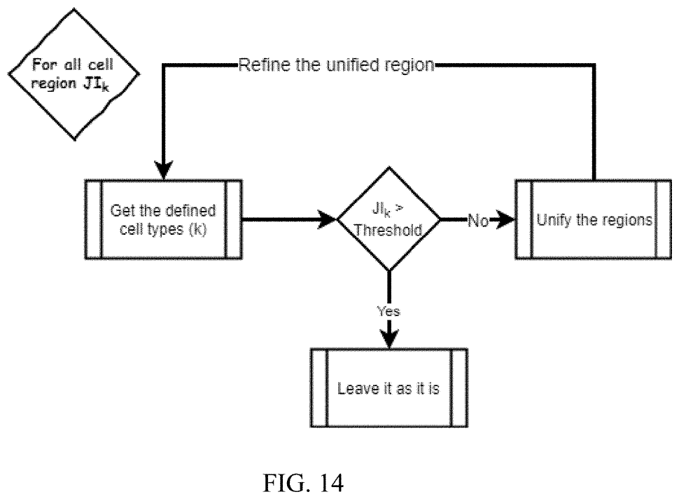

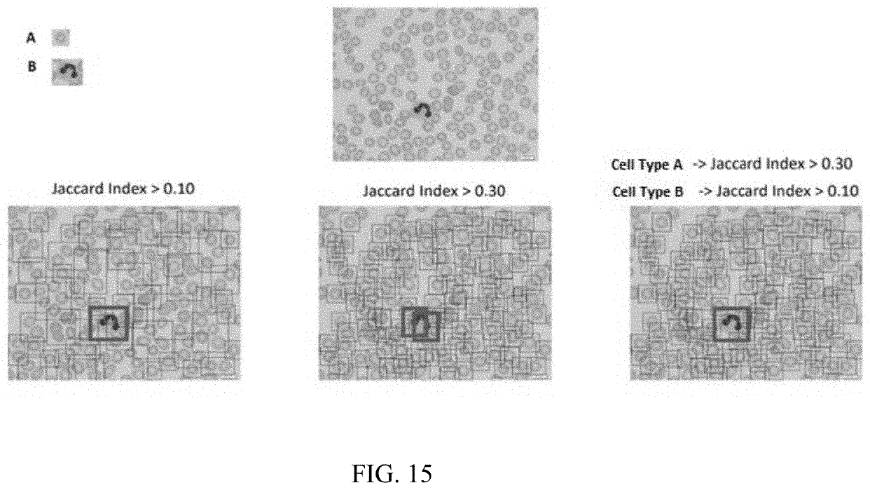

Correction is done as in FIGS. 14 and 15. After this stage, the types and the number of cell objects on the peripheral blood smear are determined as the algorithm output.

As this part is an example of an algorithm developed for peripheral blood smear, the algorithm blocks given in FIG. 4 can be developed for different types of sample types and can be included in the system. The data processing type is read by means of a barcode affixed to the biological sample slide. The cloud system knows which type of analysis to pass samples according to this barcode value.

4.2.2 Object-Based Storage of Biological Sample Images

The presented invention comprises structures that store biological samples in the cloud in a particular hierarchy and facilitate data analysis.

The storage of biological samples is organized by the hardware with the following preliminary information. Section 4.2.3 describes these steps.
IoT Password that enables pairing with the cloud.
The barcode on the biological sample is an output generated by the cloud. This barcode can be pasted onto the sample and the following informations can be extracted from the barcode by the cloud.
   Hospital ID
   Patient ID
   Analysis Type

The IoT password is unique to the physician registered on the system. As this password is given to the user over the cloud, the hardware is initialized with the help of this password and the corresponding hardware is associated with the cloud. The analysis steps are shown in FIG. 16. The flow is as follows.
1. The IOT password must be entered manually and the barcode information must be read by the barcode scanner through the sample or manually by the user for the hardware to work.
2. Each image received by the hardware is sent to the cloud) in uncompressed format with the IoT Code+Image+Checksum (Analysis Type+Control). IoT Password is a parameter to be controlled by the cloud.
   a. If this parameter cannot be matched with any hospital and physician user, the analysis code is sent to the hardware with the error code.
   b. If this parameter is associated with a physician and hospital, the algorithm software will run. The results are sent to the hardware together with the analysis report. In the analysis report, the type and position information of the objects detected on the picture are given. The analysis report content is given in FIG. 16.
3. If a successful analysis report has been generated by the cloud for all images collected by the hardware, these reports will be merged and resent for saving to the cloud.
4. 4. The data format to be thrown into the cloud is IoT Password+Barcode+1^{st} Analysis Report+2^{nd} Analysis Report+ . . . +Nth Analysis Report+Checksum (Analysis Type+Control). The data format content can be seen in FIG. 16.
5. Database is saved on the cloud using the hierarchy defined in FIG. 16. Patient information can be retrieved in this hierarchical structure as a result of necessary queries.

4.2.3 IoT Password and Barcode Encryption Method

There is a general purpose private key on the system. This private key is a valid password for the entire system. This switch is only held on C2 (Cloud).

One-way encryption infrastructure has been created for encryption, and personal, hospital or physician-specific information is only kept on the cloud. The hardware communicates with the cloud through passwords. Person, hospital or physician-specific information is not held by the hardware.

IoT Password information is generated using the following information.
Hospital ID
Doctor ID
Private Key

The cloud tests the accuracy of the password by comparing the IoT Password information sent to it with different combinations of hospitals and physicians. It continues with a correct password.

Barcode information is generated using the following information.
Hospital ID
Patient ID
Analysis Type
Private Key

The cloud tests the barcode correctness by comparing the incoming barcode information with different combinations of hospitals, patients and analysis types. If it is a correct barcode, patient number, hospital number and analysis type information will be used during the analysis process. Analysis type information is sent to the hardware for lens initialization by the cloud.

What is claimed is:

1. A method for enabling biological samples on slides to be automatically scanned and analyzed by algorithms on a cloud, comprising following steps of:
a) pairing hardware and the cloud using an Internet of Things (IOT) password;
b) placing the slides on a slide plane;
c) focusing;
d) getting a sampling number and an analysis type from the cloud;
e) analyzing the biological samples on the cloud;
f) collection of image data; and
g) hierarchical storage of the image data.

2. The method according to claim 1, wherein after step a), the slides are encoded with barcodes output from the cloud.

3. The method according to claim 2, wherein the barcodes are printed from the cloud for patients registered in the cloud.

4. The method according to claim 1, wherein the analysis type is determined manually or by a barcode.

5. The method according to claim 1, wherein an immersion oil is dripped onto the slides.

6. The method according to claim 1, wherein 10×, 40× and 100× magnification lenses are automatically selected according to the analysis type.

7. The method according to claim 1, wherein single or multiple images are collected according to the analysis type.

8. The method according to claim 1, wherein the step of focusing is manual or automatic.

9. The method according to claim 7, characterized in that it includes wherein a monitor, a keyboard and a mouse are configured for a user-controlled manual focusing.

10. The method according to claim 7, wherein a set of subjects is prepared in advance for an automatic focusing, to train a system definitions of active and noisy zones and to focus automatically at one time without a need for a learning during the step of focusing.

11. The method according to claim 1, wherein the image data is received by a user via the hardware or manually.

12. The method according to claim 11, characterized in that comprising the following steps when the image data is received via the hardware;
a) sending a biological sample image and a data processing type to an encryption and data decoder structure of a cloud system via the hardware;
b) redirecting, by the encryption and data decoder structure, the biological sample image to an algorithm adapter web service according to the data processing type;
c) running, by the algorithm adapter web service, a data processing method for the biological sample image and retrieving results from an algorithm block;
d) sending, by the encryption and data decoder structure, the results to the hardware;
e) sending, by the hardware, aggregate results to the encryption and data decoder structure together with barcode data;
f) sending by the encryption d data decoder structure, the results to a user approval via an internet interface;
g) opening the results to a user access on the cloud after the user approval.

13. The method according to claim 11, comprising the following steps when the image data is received manually by the user:
a) logging in to the cloud;
b) selecting a data processing type and a patient to associate;
c) uploading a biological sample image to a cloud system;
d) sending the biological sample image and the data processing type of the cloud system to an encryption and data decoder structure via an internet interface;
e) redirecting, by the encryption and data decoder structure, the biological sample image to an algorithm adapter web interface according to the data processing type;
f) running a data processing method for a relevant biological sample image by the algorithm adapter web interface and retrieving results from an algorithm block;
g) sending, by the encryption and data decoder structure, the results to the algorithm adapter web interface for the relevant biological sample image;
h) sending, by the encryption and data decoder structure, the results for a user approval via the internet interface; and
i) opening the results to a user access on the cloud after the user approval.

14. The method according to claim 1, wherein a property of the analysis type is a peripheral blood smear analysis, a bone marrow analysis, an analysis of a Thoma slide, a nosema illness detection at bee samples, or a lymph node analysis.

15. A system enabling automatically scanning and analyzing of the biological samples on the slide by the algorithms on the cloud, wherein the system is running with the method according to claim 1.
